(19) United States
(12) Patent Application Publication
(10) Pub. No.: US 2011/0253149 A1
(43) Pub. Date: Oct. 20, 2011

(54) APPLICATOR FOR APPLYING A BARRIER MEMBRANE TO A BODY SURFACE

(76) Inventor: Cesar Najera, La Vista, NE (US)

(21) Appl. No.: 13/086,510

(22) Filed: Apr. 14, 2011

Related U.S. Application Data
(60) Provisional application No. 61/325,935, filed on Apr. 20, 2010.

(51) Int. Cl. A61F 6/02

(52) U.S. Cl. 128/842

(57) ABSTRACT

An applicator for applying a barrier membrane to a body surface has a generally hollow body portion that holds a puncturable liquid container within. An applicator tip is provided to allow a user to apply a consistent and effective amount of liquid barrier material to a body surface, generally the tip of a penis. The liquid barrier material can include disease protection, pregnancy prevention, flavoring materials, colored films, etc. Additionally, texture enhancements such as micro-beads may be suspended within the material. The liquid barrier material is a non-toxic liquid adhesive. One embodiment uses balls to dispense the material while another embodiment uses a sponge applicator. In one embodiment, a motor is provided to automate its use. After use, the dry barrier material is peeled from the penis and discarded or is removed using a towelette containing a mixture of natural oils provided for this purpose.

### CROSS REFERENCE TO RELATED APPLICATIONS

This application claims priority and herein incorporates by reference U.S. provisional patent application 61/325,935 filed Apr. 20, 2010.

### BACKGROUND OF THE INVENTION

In today's age, the use of sexual aids is growing in both popularity and acceptance among the general population. The desire to enhance the sexual experience is as old as human nature itself. The use of physical barriers to prevent the exchange of sexual fluids is among the purposes of devices like condoms.

The first use of condoms is still debated by historians and archaeologists, but there is clear written evidence of their use in 16^{th} century Italy. Often condoms covered only the head of the penis and were used for disease control as well as pregnancy prevention. A variety of materials were used to make the condoms including animal intestine, leather, linen or other cloth material and even bone or shell. The materials were often treated with various substances to further enhance their performance.

In addition, some people use things to enhance the overall experience such as applying flavor or color to a man's penis while engaging in sexual play. While this is desirable to some, it has been generally limited to creams or a “one size fits all” covering such as a condom.

There is a need for an easy to use barrier applicator that can be used to apply a liquid barrier that allows a custom fit to enhance the sexual experience that is convenient to use and reliable even when stored for extended periods of time.

### SUMMARY OF THE INVENTION

An applicator for applying a barrier membrane to a body surface has a generally hollow body portion that holds a puncturable liquid container within. An applicator tip is provided to allow a user to apply a consistent and effective amount of liquid barrier material to a body surface, generally the tip of a penis. The liquid barrier material can include disease protection, pregnancy prevention, flavoring materials, colored films, etc. Additionally, texture enhancements such as micro-beads may be suspended within the material. The liquid barrier material is a non-toxic liquid adhesive. One embodiment uses balls to dispense the material while another embodiment uses a sponge applicator. In one embodiment, a motor is provided to automate its use. After use, the dry barrier material is peeled from the penis and discarded or is removed using a towelette containing a mixture of natural oils provided for this purpose.

Other features and advantages of the instant invention will become apparent from the following description of the invention which refers to the accompanying drawings.

### BRIEF DESCRIPTION OF THE DRAWINGS

FIG. 1 is a front view of an applicator for applying a barrier membrane to a body surface according to an embodiment of the present invention.

FIG. 2 is a detailed view of a portion shown in FIG. 1.

FIG. 3 is a front view of an applicator for applying a barrier membrane to a body surface according to an embodiment of the present invention.

FIG. 4 is a cutaway view of the applicator for applying a barrier membrane to a body surface shown in FIG. 1.

FIG. 5 is a cutaway view of the applicator for applying a barrier membrane to a body surface shown in FIG. 3.

FIG. 6 is an illustration of an applicator for applying a barrier membrane to a body surface in typical use.

FIG. 7A is a cutaway view of a motorized embodiment of the applicator for applying a barrier membrane to a body surface shown in FIG. 3 in a raised position.

FIG. 7B is a cutaway view of a motorized embodiment of the applicator for applying a barrier membrane to a body surface shown in FIG. 7A after puncture.

### DETAILED DESCRIPTION OF THE INVENTION

In the following detailed description of the invention, reference is made to the drawings in which reference numerals refer to like elements, and which are intended to show by way of illustration specific embodiments in which the invention may be practiced. It is understood that other embodiments may be utilized and that structural changes may be made without departing from the scope and spirit of the invention.

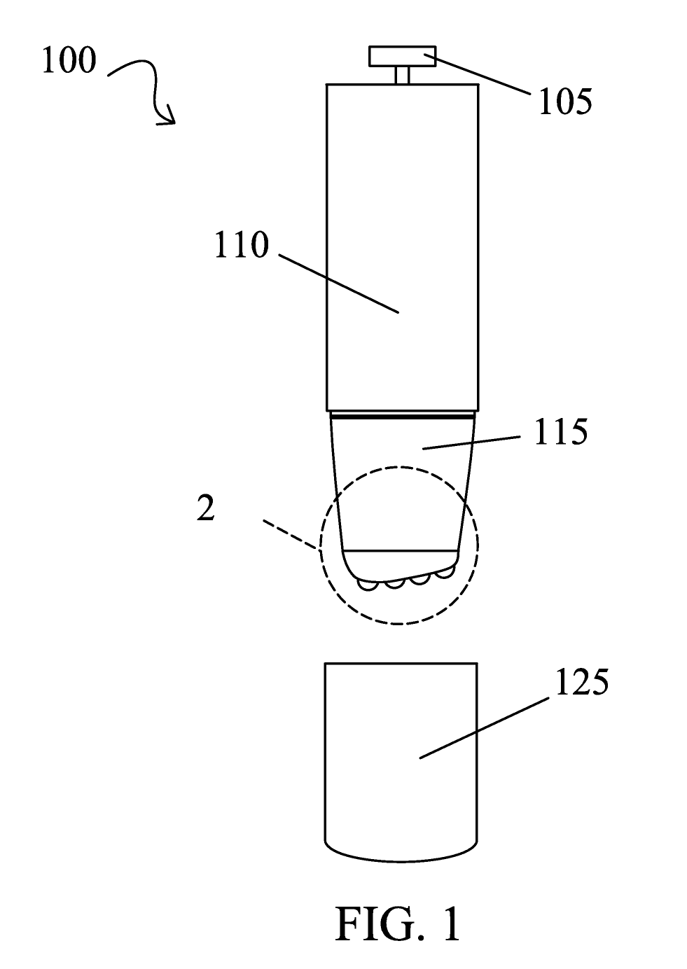

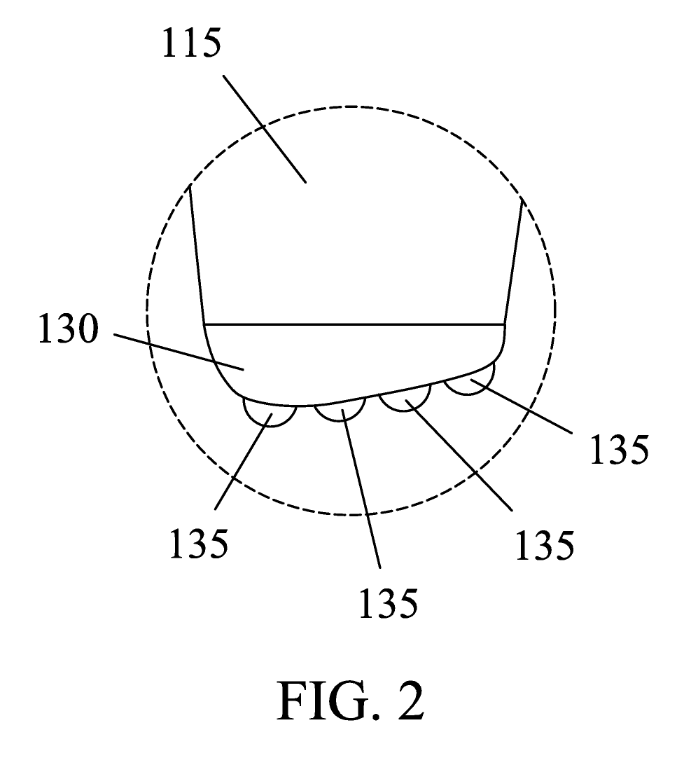

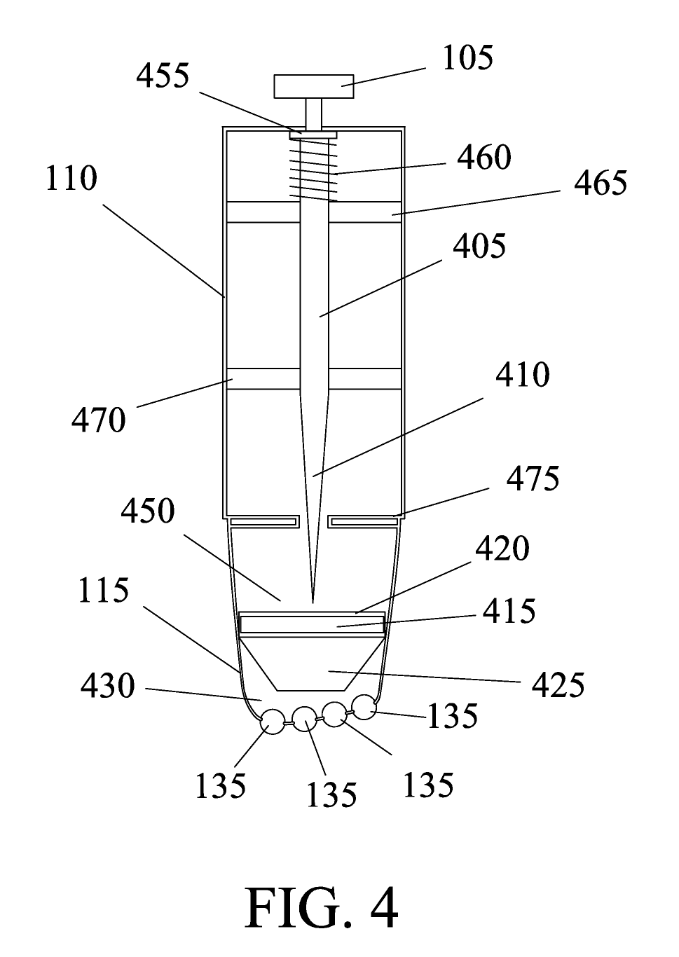

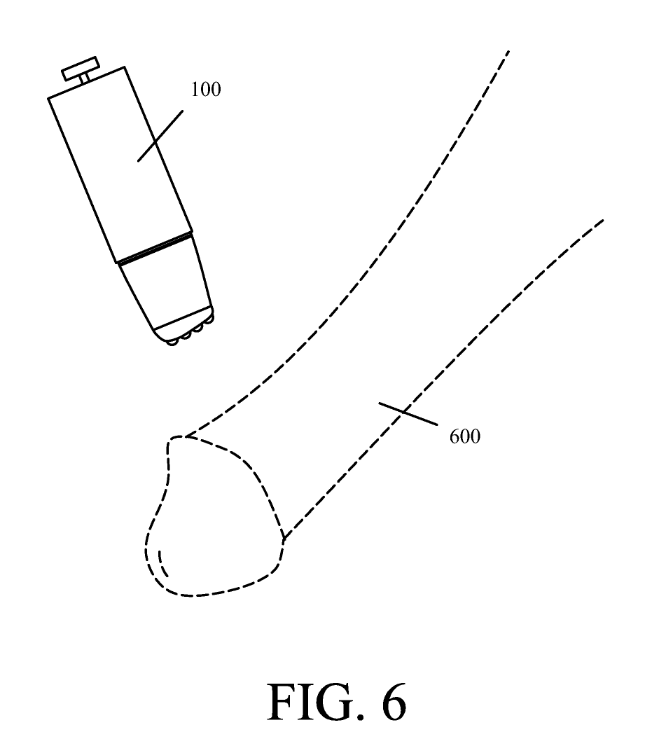

Referring to FIGS. 1, 2, 4 and 6, an applicator for applying a barrier membrane to a body surface 100 is shown having a body 110 and an applicator neck portion 115. Neck portion 115 has a dispenser tip 130 which contains a plurality of roller balls 135 which dispense an effective amount of liquid barrier material 415.

Liquid barrier material 415 is a polymeric material that adheres to skin and dries as a carrier evaporates. Some acceptable liquid adhesives and skin sealers include polymers such as polyvinylpyrrolidone (water based), pyroxylin/nitrocellulose or poly(methylacrylate-isobutene-monoisopropylmaleate) (alcohol based), and acrylate or siloxane polymers (hexamethyldisiloxane or isooctane solvent based). Of course other appropriate materials may be used as is known in the art.

Additionally, since the material is being applied to a body part, namely the tip of the penis, it is important that the material be sterile and maintained in a sterile condition until use. Hermetically sealed packaging is known in the art to provide a sealed container that will be free from contaminants until the seal is broken and is used to maintain sterility of liquid barrier material 415.

A sterile puncturable container 420 fits within neck portion 115 and neck space 450 and is punctured by pin tip portion 410 as puncture pin button 105 is depressed. Puncture pin 405 is biased by spring 460 and is retained by limiter 455 and an opening in a stiffener 465. Pin tip 410 punctures liquid container 415 allowing barrier material 415 to run down through liquid funnel portion 425 directing it toward roller balls 135. A space 430 holds a small amount of barrier material 415 thus allowing roller balls 135 to dispense a consistent amount of material as roller balls 135 are rolled across the skin around the tip of a penis 600.

A plurality of stiffeners 465, 470 and 475 are used to guide puncture pin 405 and to provide stability to liquid barrier applicator 100. Of course other methods of stiffening may be used as long as applicator 100 is sufficiently strong enough to resist crushing in use as is known in the art.

As barrier material 415 is exposed to the air, the carrier (water, alcohol or other solvent) evaporates leaving a solid yet flexible barrier in place to cover penis 600. Once dry, fluids are contained within the penis 600 and this can be important when engaging in sexual play. Barrier material 415 may include disease protection, pregnancy prevention, flavoring materials, colored films, etc.

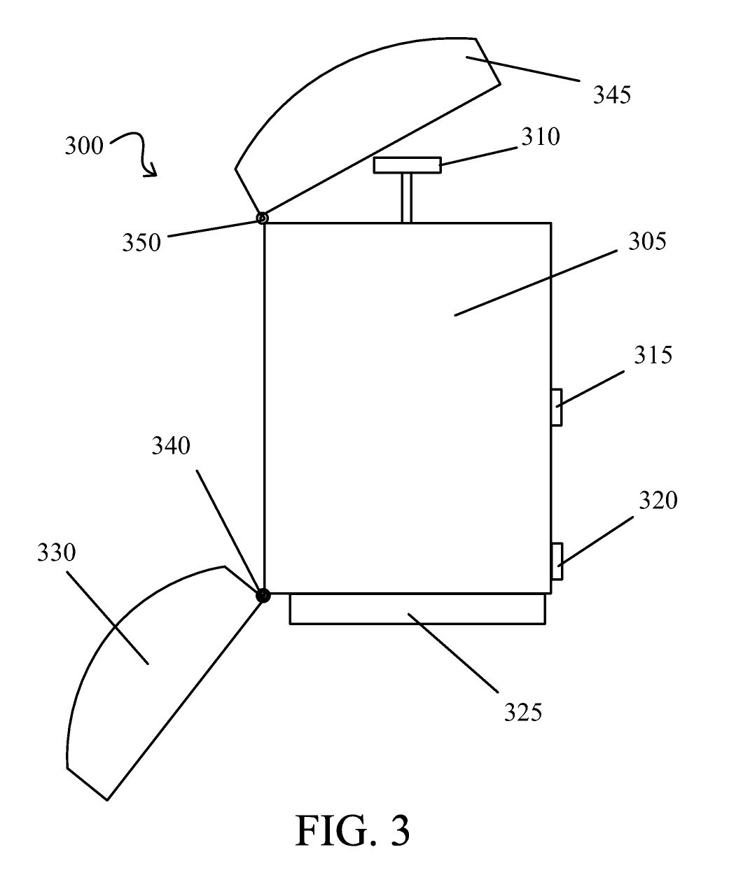

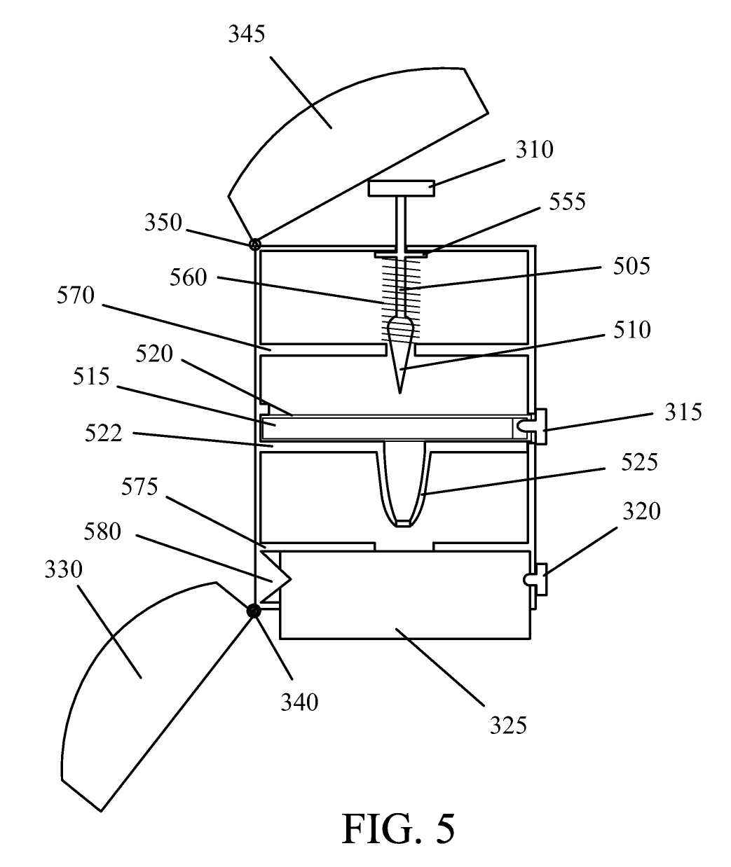

Now referring to FIGS. 3, 5 and 6, an applicator for applying a barrier membrane to a body surface 300 is shown having a body 305, a puncture pin button 310, top cap 345, bottom cap 330 and an applicator sponge 325. An upper hinge 350 rotatably holds upper cap 345 to body 305 and allows a user to open and close with use. A lower hinge 340 is similarly used to allow access applicator sponge 325. A puncture pin 505 has a pin point 510 and is used to puncture a sterile puncturable liquid container 520. A biasing spring 560 is restrained between a limiter 555 and a hole in a stiffener 570.

As pin tip 510 punctures liquid container 520, liquid barrier material 515 is released and flows through a liquid funnel portion 525 which directs is toward an opening in a lower stiffener 575 which allows barrier material 515 to disperse through sponge 325. In use, sponge 325 makes contact with the tip of penis 600 to apply an effective amount of barrier material 515.

Puncturable liquid container 520 may be replaced after use by disengaging a container retaining pin 315 and sliding used container 520 out and inserting a new one. A replacement stiffener 522 allows container 520 to slide in and out. Sponge 325 is also replace by removing a sponge holding retainer 320 and removing used sponge 325 and replacing it with a new one. A non-movable sponge retainer portion 580 is used to secure sponge 325 therein.

Additionally, in another embodiment, applicator for applying a barrier membrane to a body surface 100 is discarded after a single use. This embodiment is convenient and avoids any contamination issues associated with reuse. In this embodiment, the liquid material is maintained in a sterile condition until use and then the entire applicator is discarded.

After use, barrier material is peeled of penis 600 and discarded. Again, as discussed above, barrier material 515 may include disease protection, pregnancy prevention, flavoring materials, colored films, etc. Additionally, texture enhancements such as micro-beads may be suspended within the material. Other enhancements are possible as long as they remain in suspension in the liquid barrier material are non-toxic and inert when applied to the skin.

Alternatively, a towelette that contains a mixture of natural oils such as orange and mineral oils may be used to remove dry liquid barrier material. This mixture dissolves the dry liquid barrier material almost instantly without the use of harsh chemicals. Of course other solvents may be used as long as they are safe when applied to the skin.

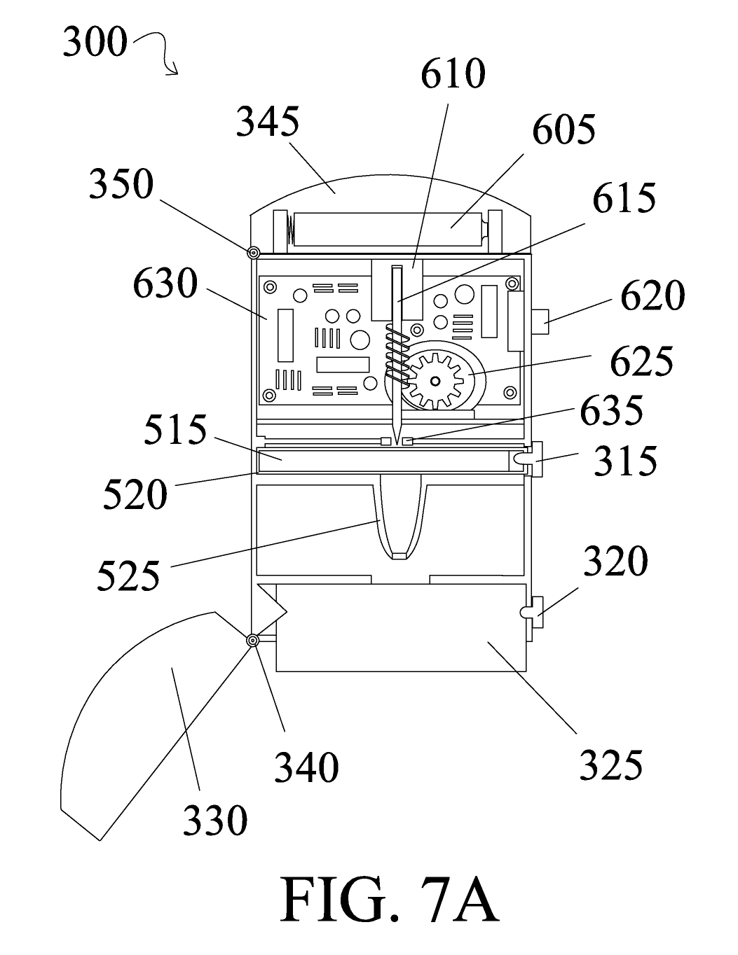

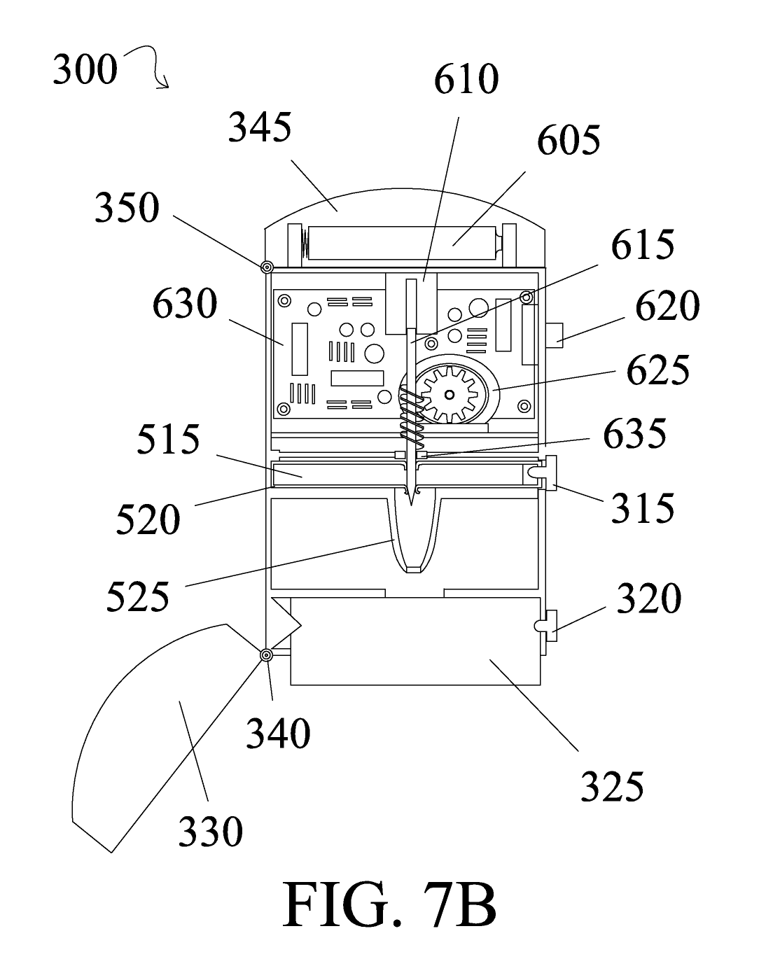

Referring now to FIGS. 7A and 7B, the applicator for applying a barrier membrane to a body surface 300 is shown having a body 305, a puncture pin button 310, top cap 345, bottom cap 330 and an applicator sponge 325. An upper hinge 350 rotatably holds upper cap 345 to body 305 and allows a user to open and replace a battery 605. A lower hinge 340 is similarly used to allow access applicator sponge 325. In this embodiment, a motor 625 is provided to move a puncture pin 615 to puncture liquid container 520 which contains liquid barrier material 515. Puncture pin 615 slide within an upper pin guide 610 and engages a lower pin guide 635. A control circuitry 630 is provided to interface between a switch 620 to move puncture pin 615 up and down. Of course other motorized embodiments would be acceptable and should be considered within the disclosure as would be known in the art.

As shown in FIG. 7B, liquid container 520 is shown punctured. As puncture pin 615 is withdrawn, liquid barrier material 515 runs out through the hole and is directed towards application sponge 325 or roller balls (see FIG. 1) as discussed above.

Although the instant invention has been described in relation to particular embodiments thereof, many other variations and modifications and other uses will become apparent to those skilled in the art.

What is claimed is:

1. An applicator for applying a barrier membrane to a body surface comprising:
a body portion being substantially hollow;
said body portion being adapted to hold a puncturable liquid container therein;
a puncture means for selectively puncturing said liquid container; and
an applicator means disposed on an end of said body portion for applying a liquid barrier material to a selected portion of said body surface.

2. The applicator for applying a barrier membrane to a body surface according to claim 1 wherein said puncturable liquid container is replaceable.

3. The applicator for applying a barrier membrane to a body surface according to claim 1 wherein said puncture means comprises a puncture pin.

4. The applicator for applying a barrier membrane to a body surface according to claim 1 wherein said applicator means comprises a sponge.

5. The applicator for applying a barrier membrane to a body surface according to claim 1 wherein said applicator means comprises at least one roller ball.

6. The applicator for applying a barrier membrane to a body surface according to claim 3 further comprising:
a motor disposed within said body; and
said motor being adapted to move said puncture pin in response to a user command.

7. The applicator for applying a barrier membrane to a body surface according to claim 1 wherein said liquid barrier material contains a flavoring.

8. The applicator for applying a barrier membrane to a body surface according to claim 1 wherein said liquid barrier material contains a spermicidal agent.

9. The applicator for applying a barrier membrane to a body surface according to claim 1 wherein said liquid barrier material contains a coloring agent.

10. The applicator for applying a barrier membrane to a body surface according to claim 1 wherein said liquid barrier material contains a textural element.

11. The applicator for applying a barrier membrane to a body surface according to claim 1 wherein said body portion is generally cylindrical.

12. The applicator for applying a barrier membrane to a body surface according to claim 1 wherein said body portion is generally rectangular.

13. The applicator for applying a barrier membrane to a body surface according to claim 1 further comprising an applicator means cap portion adapted to selectively cover said applicator means.

14. The applicator for applying a barrier membrane to a body surface according to claim 3 further comprising a puncture pin button disposed on an upper end of said body portion whereby a user can depress said puncture pin button to depress said puncture pin.

15. The applicator for applying a barrier membrane to a body surface according to claim 14 further comprising a top cap portion adapted to selectively cover said puncture pin button.

16. The applicator for applying a barrier membrane to a body surface according to claim 1 further comprising a liquid barrier material removal means for removing said liquid barrier material after use.

17. The applicator for applying a barrier membrane to a body surface according to claim 16 wherein said liquid barrier removal means is comprised of a towelette containing a mixture of natural oils.

18. The applicator for applying a barrier membrane to a body surface according to claim 17 wherein said natural oils include orange oil.

19. The applicator for applying a barrier membrane to a body surface according to claim 17 wherein said natural oils include mineral oil.

20. A method of applying a liquid barrier membrane to a body surface comprising the steps of:
obtaining an applicator for applying a barrier membrane wherein said applicator comprises;
a body portion being substantially hollow;
said body portion being adapted to hold a puncturable liquid container therein;
a puncture means for selectively puncturing said liquid container;
an applicator means disposed on an end of said body portion for applying a liquid barrier material to a selected portion of said body surface;
applying an effective amount of said liquid barrier material to a tip of a penis wherein the urethral opening is covered;
allowing said liquid barrier material to dry; and
removing said liquid barrier material after use.
